(19) United States
(12) Patent Application Publication
(10) Pub. No.: US 2009/0196944 A1
(43) Pub. Date: Aug. 6, 2009

(54) Methods of Manufacture of Morinda Citrifolia Based Compositions for Treatment of Anti-Inflammatory Diseases through Inhibition of Cox-1, Cox-2, Interleukin -1beta, Interleukin-6, TNF-alpha, HLE, and iNOS

(76) Inventors: Brad Rawson, Orem, UT (US); Matthias-Heinrich Kreuter, Walenstadt (CH); Kim Asay, Alpine, UT (US); Afa K. Palu, American Fork, UT (US); Bing-Nan Zhou, Pleasant Grove, UT (US); Brett J. West, Orem, UT (US); Claude Jarake Jensen, Cedar Hills, UT (US)

Correspondence Address: KIRTON AND MCCONKIE, 60 EAST SOUTH TEMPLE,, SUITE 1800, SALT LAKE CITY, UT, 84111, US

(21) Appl. No.: 12/024,879

(22) Filed: Feb. 1, 2008

(51) Int. Cl. A61K 36/746

(52) U.S. Cl. 424/776

(57) ABSTRACT

Methods for manufacturing compositions for inhibiting 5-Lipoxygenase, 15-Lipoxygenase are disclosed. Additionally, methods and compositions for treating and preventing diseases, including inflammatory diseases and cancer are disclosed. Compositions comprising processed Morinda citrifolia components and auxiliaries are disclosed.

### RELATED APPLICATIONS

This application claims priority to U.S. Provisional Patent Application Ser. No. 60/752,534, filed Dec. 21, 2005, and U.S. patent application Ser. No. 11/613,820, filed Dec. 20, 2006.

### BACKGROUND OF THE INVENTION

1. Field of the Invention

The present invention relates to methods of making compositions comprising Morinda citrifolia, and methods for obtaining and using the same to inhibit 5-Lipoxygenase (5-LOX) and 15-Lipoxygenase (15-LOX), COX-1, COX-2, Interleukin-1β, Interleukin-6, TNF-α, HLE, iNOS, inflammatory disease, and/or cancer.

2. Background and Related Art

Eicosanoids comprise four major classes of molecules, prostaglandins, prostacyclins, thromboxanes, and leukotrienes, and are all made mainly from arachidonic acid. Additionally, eicosanoids are continuously synthesized in membranes from 20-carbon fatty acid chains that contain at least three double bonds. The four major classes of eicosanoids are all made mainly from arachidonic acid. The synthesis of all but the leukotrienes involves the enzyme cyclooxygenase (COX); the synthesis of leukotrienes involves the enzyme lipoxygenase (LOX). Drugs are often targeted at these synthetic pathways because eicosanoids play an important part in pain, fever, and inflammation. Corticosteroid hormones such as cortisone, are examples of drugs that inhibit the activity of the phospholipase in the first step of the eicosanoid synthesis pathway and are widely used clinically to treat noninfectious inflammatory diseases, such as some forms of arthritis. Nonsteroid anti-inflammatory drugs such as aspirin and ibuprofen, by contrast, block the first oxidation step, which is catalyzed by cyclooxygenase.

In addition to inhibiting leukotrienes pathways, the inhibition of cytokines, has proven to have many clinical utilities. Cytokines are intercellular regulatory proteins that mediate various immunologic biological functions. In addition to COX, cytokines comprise Interleukin-1β (IL-1β), Interleukin-6 (IL-6), and Tumor Necosis Factor-α (TNF-α). Certain pathological disorders are attributable to unregulated cytokine production, particularly, autoimmune diseases, chronic inflammatory diseases, and some leukemias. Proper regulation of cytokines, therefore, may be desired to reduce unwanted effects of unregulated cytokine production.

Inhibition of the inducible isoform of nitric oxide (iNOS) also shows clinical benefits. Nitric oxide (NO) is synthesized from L-arginine and oxygen by NO synthase (NOS) and plays a critical role during cerebral ischemia. NO may regulate a variety of physiological functions such as blood pressure, vascular tone, permeability, and neurotransmission. Expression of iNOS leads to high output NO syntheses, which leads to cytotoxicity and inflammatory actions.

Leukotrienes are a family of lipid mediators, which are involved in acute and chronic inflammation and allergic response diseases. Leukotrienes are the biologically active metabolites of arachidonic acid. They are involved in the symptoms of inflammatory diseases, including asthma, arthritis, psoriasis, and inflammatory bowel disease. Leukotriene production starts with the oxygenation of arachidonic acid by the enzyme 5-lipoxygenase (5-LOX) into an unstable epoxide known as LTA₄. LTA₄ is an intermediate central to the formation of leukotrienes. LTA₄ may further be converted into the potent chemo attractant LTB₄ by the enzyme LTA₄ hydrolase or conjugated with glutathione (GSH) to produce LTC₄ by a specific microsomal GSH S-transferase (MGST) known as LTC₄ synthetase (LTC₄S). LTC₄ is the parent compound of the cysteinyl-leukotrienes (cys-LTs) that include LTC₄, LTD₄, and LTE₄. These three cysteinyl-leukotrienes are potent smooth muscle constricting agents, particularly in the respiratory and circulatory systems. At least two cell receptors mediate these cysteinyl-leukotrienes, CysLT1 and CysLT2. The CysLT1 receptor is a G-protein-coupled receptor with seven trans-membrane regions.

Numerous amounts of data have been collected, which clearly demonstrate that the CysLT's play a pivotal role in inflammatory and allergic response diseases, particularly asthma. The enzymes of the 5-LOX and 15-LOX pathway produce active metabolites from arachidonic acid that cause inflammation. This has been shown both by the identification of higher levels of leukotrienes in both acute and chronic inflammatory lesions coupled with the evidence of primary signs of inflammation when leukotrienes are added to tissue cultures.

In addition, the cysteinyl LT's are predominantly secreted by eosinophils, mast cells, and macrophages, which cause vasodilatation, increase postcapillary venule permeability, and stimulate bronchoconstriction and mucous secretion. Elevated leukotriene LTC₄ synthase activity was observed in peripheral blood granulocyte suspensions from patients with chronic myeloid leukemia (CML), and human bone marrow-derived myeloid progenitor cells. In asthma, the cysteinyl leukotrienes are present in alveolar lavage fluid of patients. Therefore, the presence of 5-LOX and leukotriene synthase are clinically important in the diagnosis of patients with bronchial asthma.

The lipid mediators of leukotrienes may affect cardiac output efficiency. The lipid mediators constrict coronary blood vessels, thereby reducing cardiac output. Moreover, CysLT's have been shown to induce the secretion of von Willebrand factor and surface expression of P-selectin in cultured HUVEC. Von Willebrand is a genetic disorder, the most common type of which may comprise hemophiliac diseases. The identification of higher levels of leukotrienes in both acute and chronic inflammatory lesions coupled with the evidence of primary signs of inflammation when leukotrienes are added to tissue cultures shows that enzymes of the 5-LOX pathway produce active metabolites from arachidonic acid that cause inflammation.

### SUMMARY OF THE INVENTION

Embodiments of the present invention relate to various methods of manufacturing compositions of the Indian Mulberry or Morinda citrifolia L. plant to inhibit 5-Lipoxygenase (5-LOX), 15-Lipoxygenase (15-LOX) and the lipid mediators known as leukotrienes that contribute to the pathological manifestations of inflammatory diseases, namely, asthma, arthritis, psoriasis, and inflammatory bowel disease, as well as the treatment and prevention of these diseases.

Embodiments of the invention may include one or more processed Morinda citrifolia components such as: extract from the leaves of Morinda citrifolia, leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice, whole Morinda citrifolia fruit in fresh, whole dried Morinda citrifolia fruit, powder or solvent extracted forms as well as enzyme treated Morinda citrifolia seeds, or any other processed Morinda citrifolia seed (i.e. roasting, blanching, microwaving, heat treatment, soaking in water or water solutions of various salts or chemical compounds), whole Morinda citrifolia fruit with blossoms or flowers attached, leaf extracts, leaf juice, defatted and untreated seed extracts, and Morinda citrifolia extracts processed with an auxiliary. Some of these methods include the steps of administering a Morinda citrifolia composition to a mammal to inhibit, prevent, or treat inflammatory diseases or cancer.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the above-recited and other advantages and features of the invention are understood, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIGS. 1A and 1B illustrate inhibition of 5-LOX activity by Morinda citrifolia seed extract. In particular, FIG. 1A illustrates an embodiment of inhibition with a dry extract and gummi arabicum auxiliary (sample labeled ViP_E Moci'06_{—}139.1_T). FIG. 1B illustrates inhibition with a dry extract and xanthane auxiliary (sample labeled ViP_E_Moci'06_{—}139.2_T);

FIGS. 2A and 2B illustrate inhibition of 5-LOX activity by Morinda citrifolia seed extract. In particular, FIG. 2A illustrates an embodiment of inhibition with a dry extract and Na-alginate auxiliary (sample labeled ViP_E_Moci'06_{—}139.3_T). FIG. 2B illustrates inhibition with a dry extract and guar gummi auxiliary (sample labeled ViP_E_Moci'06_{—}139.4_T).

FIGS. 3A and 3B illustrate inhibition of 5-LOX activity by Morinda citrifolia seed extract. In particular, FIG. 3A illustrates an embodiment of inhibition with a dry extract and gummi karaya auxiliary (sample labeled ViP_E_Moci'06_{—}139.5_T). FIG. 3B illustrates inhibition with a dry extract and methylcellulose auxiliary (sample labeled ViP_E_Moci'06_{—}139.6_T).

FIGS. 4A and 4B illustrate inhibition of 5-LOX activity by Morinda citrifolia seed extract, in particular FIG. 4A illustrates an embodiment of inhibition with a dry extract with no added auxiliary (sample labeled ViP_E_Moci'06_{—}139.7_T). FIG. 4B illustrates inhibition with a spissum extract and no added auxiliary (sample labeled ViP_E_Moci'06_{—}140.1).

FIGS. 5A and 5B illustrate inhibition of 5-LOX activity by Morinda citrifolia seed extract. In particular, FIG. 5A illustrates an embodiment of inhibition with a spissum extract and gummi arabicum auxiliary (sample labeled ViP_E_Moci'06_{—}140.2). FIG. 5B illustrates inhibition with a spissum extract and sterculia gummi auxiliary (sample labeled ViP_E_Moci'06_{—}140.3).

FIGS. 6A and 6B illustrate inhibition of 5-LOX activity by Morinda citrifolia seed extract. In particular, FIG. 6A illustrates an embodiment of inhibition with a spissum extract and methylcellulose auxiliary (sample labeled ViP_E_Moci'06_{—}140.4). FIG. 6B illustrates inhibition with a dry extract and gummi arabisum and aerosil auxiliary (sample labeled ViP_E_Moci'06_{—}140.5).

FIGS. 7A and 7B illustrate inhibition of 5-LOX activity by Morinda citrifolia seed extract. In particular, FIG. 7A illustrates an embodiment of inhibition with a dry extract and gummi karaya and aerosil auxiliary (sample labeled ViP_E_Moci'06_{—}140.6). FIG. 7B illustrates inhibition with a dry extract and methylcellulose and aerosil auxiliary (sample labeled ViP_E_Moci'06_{—}140.7).

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. The scope of the invention is, therefore, indicated by the appended claims rather than by the foregoing description. All changes that come within the meaning and range of equivalency of the claims are to be embraced within their scope.

Embodiments of the present invention feature methods and compositions for inhibiting, treating, and preventing mammalian inflammatory diseases and skin cancer through the administration of compositions comprising components of the Indian Mulberry or Morinda citrifolia L. plant. Additionally, some embodiments of the present invention are directed to inhibition of 5-LOX and/or 15-LOX by Morinda citrifolia L. when prepared with auxiliary agents.

### 1. GENERAL DESCRIPTION OF THE MORINDA CITRIFOLIA L. PLANT

The Indian Mulberry or Morinda citrifolia plant, known scientifically as Morinda Citrifolia L. (“Morinda citrifolia”), is a shrub or small tree up to 10 meters high. The leaves are oppositely arranged with an elliptic to ovate form. The small white flowers are contained in a fleshy, globose, head-like cluster. The fruits are large, fleshy, and ovoid. The fruit, at maturity, they is creamy-white and edible, however, they may have an unpleasant odor and/or taste. The plant is native to Southeast Asia and migrated in early times to a vast area from India to eastern Polynesia. It grows randomly in the wild, and it has been cultivated in plantations and small individual growing plots.

The Morinda citrifolia flowers are small, white, three to five lobed, tubular, fragrant, and about 1.25 cm long. The flowers develop into compound fruits composed of many small drupes fused into an ovoid, ellipsoid or round, lumpy body, with waxy, white, greenish-white, or yellowish, semi-translucent skin. Similar to a potato, the fruit contains “eyes” on its surface. The fruit is juicy, bitter, dull-yellow or yellowish-white, and contains numerous red-brown, hard, oblong-triangular, winged 2-celled stones, each containing four seeds. When fully ripe, the fruit has a pronounced odor like rancid cheese. Although the fruit has been eaten by several nationalities as food, the fruit is most commonly used as a red and yellow dye source.

### 2. PROCESSING MORINDA CITRIFOLIA LEAVES

The present invention may comprise extracts from the leaves of the Morinda citrifolia plant. For example, some compositions comprise leaf extract and/or leaf juice as described further herein. A leaf serum that is comprised of both leaf extract and fruit juice obtained from the Morinda citrifolia plant may be included in some compositions. Some compositions of the present invention comprise leaf serum and/or various leaf extracts as incorporated into a nutraceutical product (“nutraceutical” herein referring to any drug or product designed to improve the health of living organisms such as human beings or mammals).

Morinda citrifolia leaf extracts may be obtained using the following process. First, relatively dry leaves from the Morinda citrifolia L. plant are collected, cut into small pieces, and placed into a crushing device—preferably a hydraulic press—where the leaf pieces are crushed.

In some embodiments, the crushed leaf pieces are then percolated with an alcohol such as ethanol, methanol, ethyl acetate, or other alcohol-based derivatives using methods known in the art. Next, in some embodiments, the alcohol and all alcohol-soluble ingredients are extracted from the crushed leaf pieces, leaving a leaf extract that is then reduced with heat to remove all the liquid therefrom. The resulting dry leaf extract will herein be referred to as the “primary leaf extract.”

The primary leaf extract is pasteurized in some embodiments of the present invention. Pasteurization may at least partially sterilize the extract and destroy objectionable organisms. The primary leaf extract is pasteurized preferably at a temperature ranging from 70 to 80 degrees Celsius and for a period of time sufficient to destroy any objectionable organisms without major chemical alteration of the extract. Pasteurization may also be accomplished according to various radiation techniques or methods.

The pasteurized primary leaf extract may be placed into a centrifuge decanter, in some embodiments of the present invention, where it may be centrifuged to remove or separate any remaining leaf juice therein from other materials, including chlorophyll. Once the centrifuge cycle is completed, the leaf extract is in a relatively purified state. This purified leaf extract may then be pasteurized again in a similar manner to that discussed above to obtain a purified primary leaf extract.

Additionally, the primary leaf extract, whether pasteurized and/or purified, may be further fractionated into two individual fractions: a dry hexane fraction, and an aqueous methanol fraction. This is accomplished preferably via a gas chromatograph containing silicon dioxide and CH₂Cl₂-MeOH ingredients using methods well known in the art. In some embodiments of the present invention, the methanol fraction is further fractionated to obtain secondary methanol fractions. In some embodiments, the hexane fraction is further fractionated to obtain secondary hexane fractions.

One or more of the leaf extracts, including the primary leaf extract, the hexane fraction, methanol fraction, or any of the secondary hexane or methanol fractions may be combined with the fruit juice of the fruit of the Morinda citrifolia plant to obtain a leaf serum (the process of obtaining the fruit juice to be described further herein). In some embodiments, the leaf serum is packaged and frozen ready for shipment; in others, it is further incorporated into a nutraceutical product as explained herein.

### 3. PROCESSING MORINDA CITRIFOLIA FRUIT

The fruit juice of the Morinda citrifolia plant my be included in some embodiments of the present invention. Because may people find the Morinda citrifolia fruit inedible, the fruit may be processed in order to make it palatable for human consumption and for inclusion in the compositions of the present invention.

Morinda citrifolia fruit juice may be prepared by separating seeds and peels from the juice and pulp of ripe Morinda citrifolia fruit. The pulp may then be filtered from the juice, and the juice may then be packaged. Rather than packaging the juice, the juice may be frozen or pasteurized and can be used immediately as an ingredient in another product. In some embodiments of the present invention, the juice and pulp can be pureed into a homogenous blend to be mixed with other ingredients. The fruit and juice may be freeze dried in some embodiments of the invention. The fruit and juice can be reconstituted during production of the final juice product. Other processes may include air drying the fruit and juices.

One current process for production of Morinda citrifolia fruit juice will now be described. The Morinda citrifolia fruit may be either hand picked or picked by mechanical equipment. The fruit can be harvested when it is at least one inch (2-3 cm) and up to 12 inches (24-36 cm) in diameter. The fruit preferably is dark green to yellow-green or white in color when harvested. The fruit may be thoroughly cleaned after harvesting, before any processing occurs. The fruit may be allowed to ripen or age for 2 to 3 days but may ripen between 0 to 14 days. The fruit is preferably covered with a cloth or netting material during aging, but the fruit can be aged without being covered. When ready for further processing the fruit is light in color, such as a light green, light yellow, white or translucent color. The fruit is inspected, and spoiled and hard green fruit is separated from the acceptable fruit.

The aged fruit can be held from 0 to 30 days, but preferably the fruit is held for 7 to 14 days before processing. The fruit can optionally be stored under refrigerated conditions prior to further processing. The fruit is processed through a manual or mechanical separator, and the seeds and peel are separated from the juice and pulp. The juice and pulp may be stored, or the juice and pulp may be immediately processed into a finished juice product. The juice and pulp may be stored in refrigerated, frozen, or room temperature conditions. The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

The juice and pulp may be separated through filtering equipment. The filtering equipment preferably consists of, but is not limited to, a centrifuge decanter, a screen filter with a size from 1 micron up to 2000 microns, more preferably less than 500 microns, a filter press, a reverse osmosis filtration device, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered between 1 and 10 times to remove any juice from the pulp. The resulting pulp extract typically has a fiber content of 10 to 40 percent by weight. The resulting pulp extract is preferably pasteurized at a temperature of 181° F. (83° C.) minimum and then packed in drums for further processing or made into a high fiber product.

Another product that may be manufactured is Morinda citrifolia puree and puree juice, in either concentrate or diluted form. Puree is essentially the pulp separated from the seeds and is different than the fruit juice product described herein.

Morinda citrifolia product may be filled and sealed into a final container of plastic, glass, or another suitable material that can withstand processing temperatures. The containers may be maintained at the filling temperature or may be cooled rapidly and then placed in a shipping container. The shipping containers are preferably wrapped with a material in a manner to maintain or control the temperature of the product in the final containers.

### 4. PROCESSING MORINDA CITRIFOLIA SEEDS

Morinda citrifolia extracts may be prepared from seeds of the Morinda citrifolia plant for inclusion in some embodiments of the present invention. Morinda citrifolia seeds may be processed by pulverizing them into a seed powder using a laboratory mill. The seed powder may be left untreated. Alternatively, the seed powder may be defatted by soaking and stirring the powder in hexane—preferably for 1 hour at room temperature (Drug:Hexane—Ratio 1:10). The residue, in some embodiments, is then filtered under vacuum. The filtered powder may be defatted again (preferably for 30 minutes under the same conditions), and filtered under vacuum again. The powder is preferably kept overnight in a fume hood in order to remove the residual hexane. Additionally, the defatted and/or untreated powder may be extracted, preferably with ethanol 50% (m/m) for 24 hours at room temperature at a drug solvent ratio of 1:2.

### 5. PROCESSING MORINDA CITRIFOLIA OIL

Some embodiments of the present invention may comprise oil extracts from the Morinda Citrifolia plant. The method for extracting and processing the oil is described in U.S. patent application Ser. No. 09/384,785, filed on Aug. 27, 1999 and issued as U.S. Pat. No. 6,214,351 on Apr. 10, 2001, which is incorporated by reference herein. Morinda citrifolia oil comprises several different fatty acids as triglycerides, such as palmitic, stearic, oleic, and linoleic fatty acids, and other fatty acids present in lesser quantities. The oil may include an antioxidant to inhibit spoilage. When antioxidants are included, conventional food grade antioxidants are preferably used.

### 6. COMPOSITIONS AND THEIR USE

The present invention features compositions and methods for inhibiting 5-LOX, 15-LOX, and/or skin cancer. The present invention also features compositions and methods for inhibiting the oxygenation of arachidonic acid into its leukotriene intermediate constituents for the purpose of treating and preventing inflammatory diseases. Embodiments of the present invention also comprise methods for internally introducing a Morinda citrifolia composition into the body of a mammal. Several embodiments of the Morinda citrifolia compositions comprise various different ingredients, each embodiment comprising one or more forms of a processed Morinda citrifolia component as taught and explained herein.

Compositions of the present invention may comprise any number of Morinda citrifolia components such as: leaf extract, leaf juice, leaf serum, fruit juice, fruit pulp, pulp extract, puree, seeds (whether defatted or untreated), and oil. Compositions of the present invention may also include various other ingredients, such as: artificial flavoring, other natural juices or juice concentrates such as a natural grape juice concentrate or a natural blueberry juice concentrate, carrier ingredients, auxiliaries, and others as will be further explained herein. Any compositions having the Morinda citrifolia leaf extract may comprise one or more of the following: primary leaf extract, hexane fraction, methanol fraction, secondary hexane and methanol fractions, leaf serum, or nutraceutical leaf product.

In some embodiments of the present invention, active ingredients or compounds of Morinda citrifolia components may be extracted using various procedures and processes commonly known in the art. For instance, the active ingredients may be isolated and extracted using alcohol or alcohol-based solutions, such as methanol, ethanol, and ethyl acetate, and other alcohol-based derivatives using methods known in the art. These active ingredients or compounds may be isolated and further fractioned or separated from one another into their constituent parts. Preferably, the compounds are separated or fractioned to identify and isolate any active ingredients that might help to prevent disease, enhance health, or perform other similar functions. In addition, the compounds may be fractioned or separated into their constituent parts to identify and isolate any critical or dependent interactions that might provide the same health-benefiting functions just mentioned.

Components and compositions of Morinda citrifolia may be further incorporated into a nutraceutical product. Nutraceutical products may include, but are not limited to: intravenous products, topical dermal products, wound healing products, skin care products, hair care products, beauty and cosmetic products (e.g., makeup, lotions, etc.), burn healing and treatment products, first-aid products, antibacterial products, lip balms and ointments, bone healing and treatment products, meat tenderizing products, anti-inflammatory products, eye drops, deodorants, antifungal products, arthritis treatment products, muscle relaxers, toothpaste, and various nutraceutical and other products as may be further discussed herein.

The compositions of the present invention may be formulated into any of a variety of substances, including oral compositions, topical dermal solutions, intravenous solutions, and other products or compositions.

Oral compositions may include, for example, tablets, lozenges, aqueous or oily suspensions, dispersible powders or granules, emulsions, syrups, or elixirs. Oral compositions may be prepared according to any method known in the art and may contain one or more agents such as sweetening agents, flavoring agents, coloring agents, and preserving agents. Oral compositions may also contain additional ingredients such as vitamins and minerals, etc. Morinda citrifolia components in admixture with non-toxic, pharmaceutically acceptable excipients may be used in the manufacture of tablets. Excipients may be, for example, inert diluents, granulating and disintegrating agents, binding agents, and lubricating agents. Tablets may be uncoated or ma y be coated by known techniques to delay disintegration and absorption in the gastrointestinal tract and thereby provide sustained action over a longer period. For example, a time delay material such as glyceryl monostearate or glyceryl distearate may be used.

Morinda citrifolia components in admixture with excipients may be used in the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Sweetening agents may be included in some embodiments of the present invention. Typical sweetening agents include, but are not limited to: natural sugars derived from corn, sugar beets, sugar cane, potatoes, tapioca, or other starch-containing sources that can be chemically or enzymatically converted to crystalline chunks, powders, and/or syrups. Also, sweeteners can comprise artificial or high-intensity sweeteners, some of which may include aspartame, sucralose, stevia, saccharin, etc. Sweeteners may comprise between from 0 to 50 percent by weight of the Morinda citrifolia composition, and more preferably between about 1 and 5 percent by weight. Some embodiments of the present invention may comprise flavoring and/or coloring agents. Flavoring agents may include, but are not limited to, artificial and/or natural flavoring ingredients that contribute to palatability. Flavorant concentration may range, for example, from 0 to 15 percent by weight of the Morinda citrifolia composition. Coloring agents may include food-grade artificial or natural coloring agents and may be concentrated from 0 to 10 percent by weight of a Morinda citrifolia composition.

Various nutritional ingredients may be included in some embodiments of the present invention. Nutritional ingredients may include vitamins, minerals, trace elements, herbs, botanical extracts, bioactive chemicals, and compounds at concentrations from 0 to 10 percent by weight of the Morinda citrifolia composition. Examples of vitamins include, but are not limited to, vitamins A, B1 through B12, C, D, E, Folic Acid, Pantothenic Acid, Biotin, etc. Minerals and trace elements include, but are not limited to, calcium, chromium, copper, cobalt, boron, magnesium, iron, selenium, manganese, molybdenum, potassium, iodine, zinc, phosphorus, etc. Examples of herbs and botanical extracts may include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

Topical dermal products may include any ingredients that are safe for internalizing into the body of a mammal. Such ingredients may include, but are not limited to, gels, lotions, creams, ointments, etc., each comprising one or more carrier agents. Systemically administered compositions may comprises any ingredients or carriers know in the art.

Several embodiments of formulations of the invention are included in U.S. Pat. No. 6,214,351, issued on Apr. 10, 2001. However, these compositions are only intended to be exemplary, as one ordinarily skilled in the art will recognize other formulations or compositions comprising the processed Morinda citrifolia product.

Some embodiments of the invention comprise processed Morinda citrifolia fruit juice or puree juice present in an amount by weight between about 0.1-80 percent, processed Morinda citrifolia oil present in an amount by weight between about 0.1-20 percent, and a carrier medium present in an amount by weight between about 20-90 percent. Morinda citrifolia puree juice or fruit juice may also be formulated with a processed Morinda citrifolia dietary fiber product present in similar concentrations.

### 7. EXAMPLES

The following examples illustrate some of the preventative and treatment effects of some Morinda citrifolia compositions of the present invention on 5-LOX, 15-LOX, COX-1, COX-2, Interleukin 1β, Interleukin-6, TNF-α, HLE, iNOS, inflammatory diseases, and/or cancer. These examples are not intended to be limiting in any way, but are merely illustrative of benefits, advantages, and remedial effects of some embodiments of the Morinda citrifolia compositions of the present invention.

### Example 1

A study was performed to measure the inhibitory effects of Morinda citrifolia seed extracts on the activity of human 5-Lipoxygenase (5-LOX) when processed under a variety of conditions and with a variety of auxiliaries. A fluid extract was prepared from dry Morinda citrifolia seeds at 60° C. with 80% ETOH w/w and with a drug solvent ratio of 1:6. The fluid extract was separated into different samples. To these samples, different auxiliaries were added, and the samples were concentrated to a spissum extract at 40° C. Table 1 summarizes the fluid extract preparations.

TABLE 1
Spissum samples

| | | Auxiliary | |
|---|---|---|---|
| Sample ID | Auxiliary | (%) | Extract Type |
| ViP_E_Moci′06_139.1 | Gummi Arabicum | 29 | Spissum |
| ViP_E_Moci′06_139.2 | Xanthane | 29 | Spissum |
| ViP_E_Moci′06_139.3 | Na-Alginate | 29 | Spissum |
| ViP_E_Moci′06_139.4 | Guar Gummi | 29 | Spissum |
| ViP_E_Moci′06_139.5 | Gummi Karaya | 29 | Spissum |
| ViP_E_Moci′06_139.6 | Methylcellulose | 29 | Spissum |
| ViP_E_Moci′06_139.7 | | | Spissum |
| ViP_E_Moci′06_140.1 | | | Spissum |
| ViP_E_Moci′06_140.2 | Gummi Arabicum | 29 | Spissum |
| ViP_E_Moci′06_140.3 | Sterculia gummi | 29 | Spissum |
| ViP_E_Moci′06_140.4 | Methylcellulose | 29 | Spissum |

The spissum samples were then dried to a dry extract, applying different drying temperatures. The dry extract samples are summarized in Table 2.

TABLE 2
Dry extract samples

| | | Auxiliary | Extract | Drying |
|---|---|---|---|---|
| Sample ID | Auxiliary | (%) | Type | Temp (° C.) |
| ViP_E_Moci′06_139.1_T | Gummi Arabicum | 29 | Dry extract | 40 |
| ViP_E_Moci′06_139.2_T | Xanthane | 29 | Dry extract | 40 |
| ViP_E_Moci′06_139.3_T | Na-Alginate | 29 | Dry extract | 40 |
| ViP_E_Moci′06_139.4_T | Guar Gummi | 29 | Dry extract | 40 |
| ViP_E_Moci′06_139.5_T | Gummi Karaya | 29 | Dry extract | 40 |
| ViP_E_Moci′06_139.6_T | Methylcellulose | 29 | Dry extract | 40 |
| ViP_E_Moci′06_139.7_T | | | Dry extract | 40 |
| ViP_E_Moci′06_140.5 | Gummi Arabicum | 29 | Dry extract | 55 |
| | Aerosil | 1 | | |
| ViP_E_Moci′06_140.6 | Gummi Karaya | 29 | Dry extract | 55 |
| | Aerosil | 1 | | |
| ViP_E_Moci′06_140.7 | Methylcellulose | 29 | Dry extract | 55 |
| | Aerosil | 1 | | |

A Lipoxygenase Assay in human HL-60 cells was then performed as follows. Human HL-60 cells (myeloid leukemia, DSMZ No ACC 3) were kept at 37° C. in a humidified atmosphere with 5% CO₂ and cultured in complete RPMI 1640 medium supplemented with 10% fetal calf serum and 1% (v/v) penicillin/streptomycin solution. Cells were differentiated for 6 to 8 days with DMSO (1.2% v/v). The 5-LOX activity assay was carried out as described by C. F. Bennet, M. Y. Chiang, B. P. Monia, and S. T. Crooke in “Regulation of 5-lipoxygenase-activating protein expression in HL-60 cells,” Biochem. J. 289: 33-39. Briefly, differentiated cells were harvested, suspended in PBS containing Ca²⁺ (1 mM) and glucose (1 mM) and distributed into a 96-well microtiter plate (1×10⁶ cells/well).

Stock solutions of test compounds in appropriate solvent were diluted with PBS. After pre-incubation with a sample or vehicle for 15 minutes at room temperature, the reaction was started by adding calcium ionophore A 23187 (5 μM) and arachidonic acid (10 μM). All values taken represented final values for the solvent concentrations. Negative controls were carried out without calcium ionophore stimulation. The assay mix (100 μl) was incubated for 15 minutes at 37° C. and terminated by adding 100 μl methanol containing HCl (1 M, 3% v/v) and placing the microtiter plate on ice. After centrifugation (340×g) for 10 minutes, the LTB₄ concentration in the supernatant was determined.

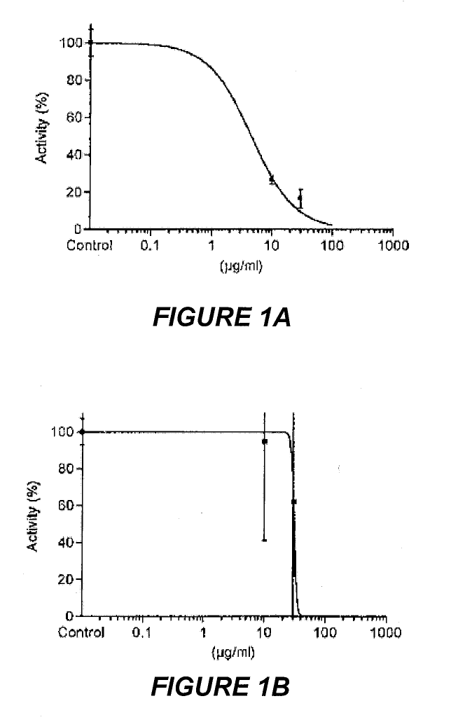

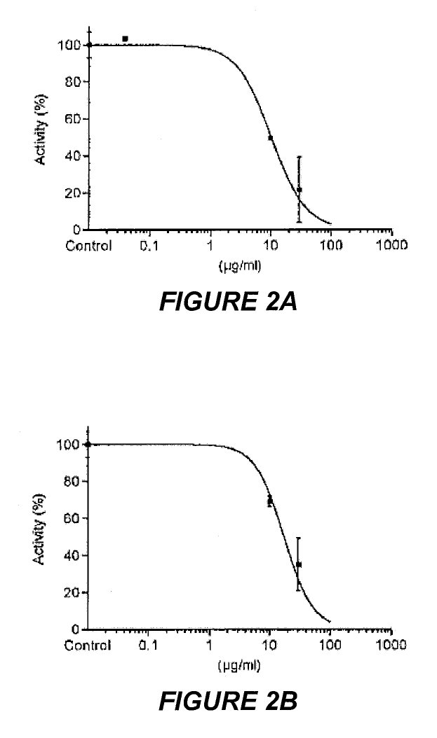

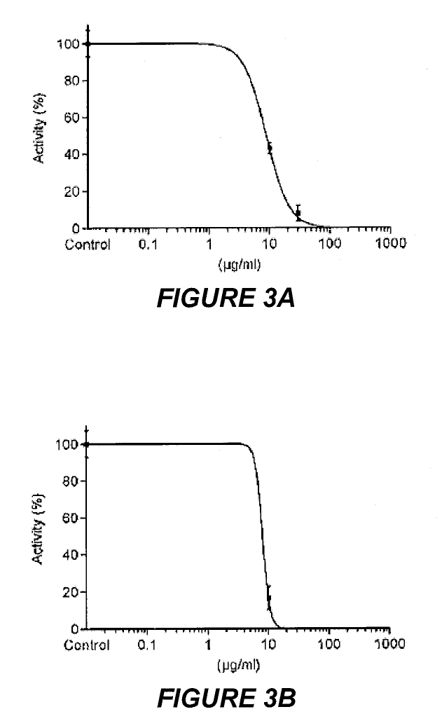

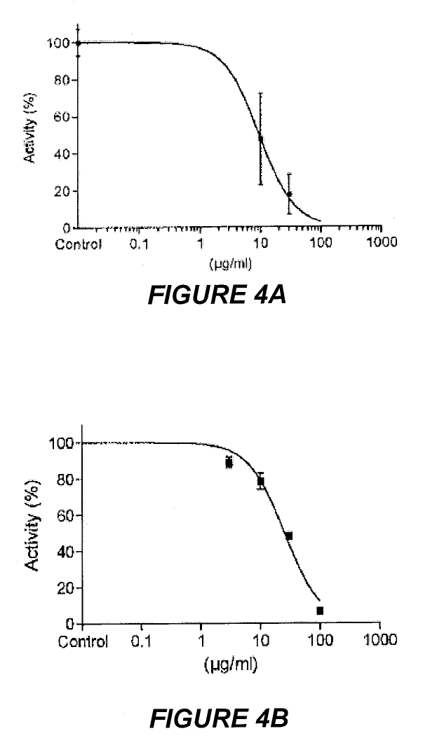

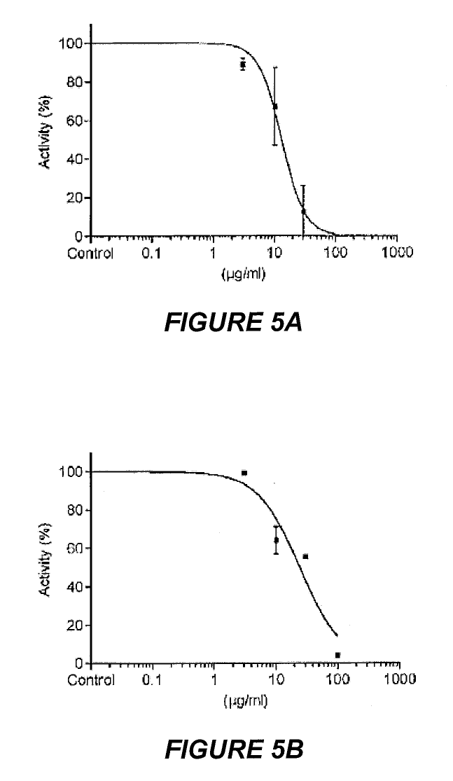

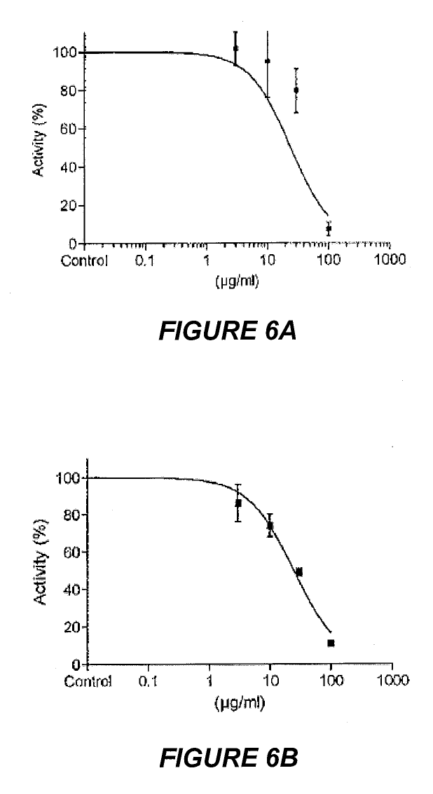

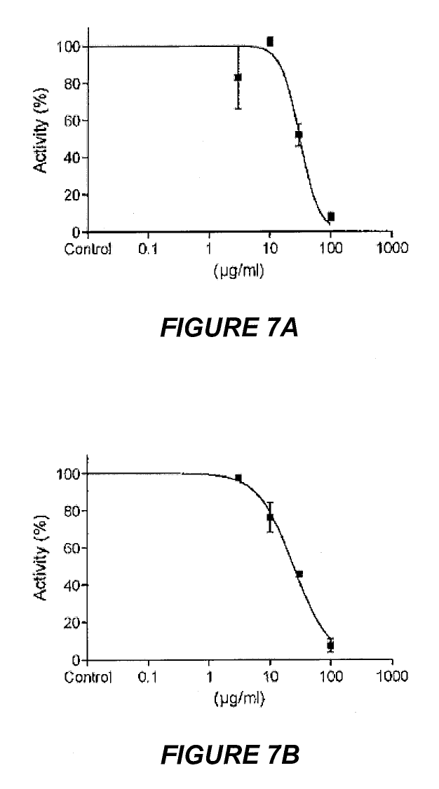

Effects of samples and reference compounds on the activity of 5-LOX were measured by determining the quantity of Leukotrien B₄ produced under assay conditions. The quantification of Leukotrien B₄ was performed with Enzyme Immuno Assay (EIA) Kit from Cayman No 520111 (LTB₄). The optical densities were measured at λ=415 nm. The quantities were calculated using a standard curve of at least 5 different concentrations. Sample points were measured as duplicates. Dose related inhibition values were expressed as a percentage of the positive control values. The following tables and charts and FIGS. 1A through 7B summarize the assay and the results.

TABLE 3
IC₅₀ Values

| Sample | Auxiliaries | IC₅₀ (μg/ml) | 95% Interval |
|---|---|---|---|
| ViP_E_Moci′06_139.1_T | 29% Gummi arabicum | 4.5 | 1.1-18.6 |
| ViP_E_Moci′06_139.2_T | 29% Xanthan | 30.8 | *na. |
| ViP_E_Moci′06_139.3_T | 29% Na-Alginate | 10.25 | 5.5-19.2 |
| ViP_E_Moci′06_139.4_T | 29% Guar Gummi | 17.4 | 10.5-28.8 |
| ViP_E_Moci′06_139.5_T | 29% Sterculia Gummi | 8.9 | 6.1-13.0 |
| ViP_E_Moci′06_139.6_T | 29% Methylcellulose | 8.1 | *na. |
| ViP_E_Moci′06_139.7_T | | 9.5 | 4.8-18.7 |
| ViP_E_Moci′06_140.1 | | 25.3 | 18.9-34.0 |
| ViP_E_Moci′06_140.2 | 29% Gummi arabicum | 13.2 | 8.3-20.8 |
| ViP_E_Moci′06_140.3 | 29% Sterculia Gummi | 24.1 | 13.9-42.0 |
| ViP_E_Moci′06_140.4 | 29% Methylcellulose | 45.8 | 28.1-74.8 |
| ViP_E_Moci′06_140.5 | 29% Gummi arabicum | 24.6 | 16.61-36.32 |
| | 1% Aerosil | | |
| ViP_E_Moci′06_140.6 | 29% Sterculia Gummi | 31.5 | 22.5-44.0 |
| | 1% Aerosil | | |
| ViP_E_Moci′06_140.7 | 29% Methylcellulose | 24.4 | 18.5-32.2 |
| | 1% Aerosil | | |
| Standard | | IC₅₀ (μM) | 95% Interval |
| NDGA | | 0.1-0.7 | *na. |

*na. not applicable

TABLE 4
Raw data of 5-LOX inhibition

| | t(15) | t(0) | | t(15) | t(0) |
|---|---|---|---|---|---|
| Concentration | LTB4 | LTB4 | Concentration | LTB4 | LTB4 |
| (μg/ml) | (pg/ml) | (pg/ml) | (μg/ml) | (pg/ml) | (pg/ml) |
| ViP_E_Moci′06_139.1_T | | | ViP_E_Moci′06_139.2_T | | |
| 100 | 1283 | 1484 | 100 | 1209 | 1792 |
| 100 | 1104 | | 100 | 1382 | |
| 30 | 2075 | | 30 | 2148 | |
| 30 | 1891 | | 30 | 5103 | |
| 10 | 2253 | | 10 | 3435 | |
| 10 | 2348 | | 10 | 5701 | |
| Control | 4668 | 1545 | Control | 4668 | 1545 |
| Control | 4383 | | Control | 4393 | |
| ViP_E_Moci′06_139.3_T | | | ViP_E_Moci′06_139.4_T | | |
| 100 | | 2033 | 100 | 1029 | 1706 |
| 100 | 1478 | | 100 | 1422 | |
| 30 | 2998 | | 30 | 2440 | |
| 30 | 2229 | | 30 | 3023 | |
| 10 | 3385 | | 10 | 3653 | |
| 10 | 3389 | | 10 | 3786 | |
| Control | 4668 | 1545 | Control | 4668 | 1545 |
| Control | 4383 | | Control | 4393 | |
| ViP_E_Moci′06_139.5_T | | | ViP_E_Moci′06_139.6_T | | |
| 100 | 1493 | 1944 | 100 | 1285 | 2012 |
| 100 | 1409 | | 100 | 1327 | |
| 30 | 2044 | | 30 | 1736 | |
| 30 | 2234 | | 30 | 1375 | |
| 10 | 3064 | | 10 | 2534 | |
| 10 | 3182 | | 10 | 2253 | |
| Control | 4668 | 1545 | Control | 4668 | 1545 |
| Control | 4383 | | Control | 4393 | |
| ViP_E_Moci′06_139.7_T | | | ViP_E_Moci′06_140.1 | | |
| 100 | 1316 | 1766 | 100 | 445 | 292 |
| 100 | 2109 | | 100 | 458 | |
| 30 | 2493 | | 30 | 1713 | 226 |
| 30 | 2046 | | 30 | 1600 | |
| 10 | 2601 | | 10 | 2388 | |
| 10 | 3666 | | 10 | 2650 | |
| Control | 4668 | 1545 | 3 | 2733 | |
| Control | 4383 | | 3 | 2906 | |
| | | | Control | 2782 | 234 |
| | | | Control | 3474 | |
| ViP_E_Moci′06_140.2 | | | ViP_E_Moci′06_140.3 | | |
| 100 | 1264 | 1600 | 100 | 1421 | 1436 |
| 100 | 1199 | | 100 | 1452 | |
| 30 | 2033 | 1137 | 30 | 2623 | 1244 |
| 30 | 1416 | | 30 | 2597 | |
| 10 | 2502 | | 10 | 2978 | |
| 10 | 3425 | | 10 | 2653 | |
| 3 | 3547 | | 3 | 3621 | |
| 3 | 3399 | | 3 | | |
| Control | 3280 | 1449 | Control | 3280 | 1449 |
| Control | 4125 | | Control | 4125 | |
| ViP_E_Moci′06_140.4 | | | ViP_E_Moci′06_140.5 | | |
| 100 | 1284 | 1355 | 100 | 540 | 249 |
| 100 | 1453 | | 100 | 601 | |
| 30 | 3294 | 1031 | 30 | 1623 | 273 |
| 30 | 2773 | | 30 | 1729 | |
| 10 | 3854 | | 10 | 2566 | |
| 10 | 2973 | | 10 | 2235 | |
| 3 | 3787 | | 3 | 3018 | |
| 3 | 3382 | | 3 | 2465 | |
| Control | 3280 | 1449 | Control | 2782 | 234 |
| Control | 4125 | | Control | 3474 | |
| ViP_E_Moci′06_140.6 | | | ViP_E_Moci′06_140.7 | | |
| 100 | 457 | 306 | 100 | 439 | 354 |
| 100 | 584 | | 100 | 639 | |
| 30 | 1611 | 277 | 30 | 1612 | 281 |
| 30 | 1969 | | 30 | 1634 | |
| 10 | 3181 | | 10 | 2276 | |
| 10 | 3284 | | 10 | 2729 | |
| 3 | 2201 | | 3 | 3144 | |
| 3 | 3181 | | 3 | 3113 | |
| Control | 2782 | 234 | Control | 2782 | 234 |
| Control | 3474 | | Control | 3474 | |

In summary, the calculated IC₅₀ values including the 95% interval, as summarized in Table 3, demonstrate that the developed process conditions are superior compared to others. With an IC₅₀ value of 4.5 μg/ml the dry extract ViP_E_Moci'06_{—}139.1_T (Arabic Gum) is clearly the best dry extract of all tested products. Nevertheless, the dry extracts produced with methylcellulose (ViP_E_Moci'06_{—}139.6_T) and with Sterculia Gum (ViP_E_Moci'06_{—}139.5_T) are, in principle, acceptable but they don't exceed the activity of the native extract (ViP_E_Moci'06_{—}139.7_T). As Sterculia Gum (also called Gum Karaya) is sticky and adhesive it is not the first choice for the drying process, as it makes the handling of the extract complicated. Methylcellulose becomes extremely viscous, and, therefore, it is hard to evaporate the remaining water that makes the drying process complicated and expensive. This is even more important as we see a significant loss in activity in samples dried at 55° C. in respect to those dried at 40° C. (compare ViP_E_Moci'06_{—}140.5 with ViP_E_Moci'06_{—}139.1_T). In summary, we see clear superiority of Arabic Gum used as an auxiliary with a Morinda citrifolia dry extract.

### Example 2

In another example, a Morinda citrifolia extract may be dried at temperatures other than 40° C. or 55° C. For example a Morinda citrifolia extract may be dried at any temperature between 25° C. and 100° C. One skilled in the art will recognize that a variety of drying temperatures may be used consistent with the invention.

Although auxiliaries comprising about 30% of the Morinda citrifolia based composition were used with reference to Example 1, in some embodiments an auxiliary may be present in varying amounts in the Morinda citrifolia composition. For example, an auxiliary that is combined with a Morinda citrifolia extract may comprise 5%, 10%, 25%, 30%, 35%, or 50% of a Morinda citrifolia composition.

Further, a Morinda citrifolia composition may also comprise reagents other than auxiliaries and Morinda citrifolia. For example, a flavoring may be combined with a Morinda citrifolia composition. A colorant may also be combined with a Morinda citrifolia composition. A Morinda citrifolia composition may comprise other therapeutic reagent known in the art. Many different and varied additives and reagents that may be combined with a Morinda citrifolia composition that are consistent with the spirit of the invention.

### Example 3

The following is a preferred example of a method of manufacturing Morinda citrifolia products into compositions, which may be utilized to decrease inflammation, inhibit various enzymes, and may additionally be utilized to prevent, treat or ameliorate cancer. In the present non-limiting example, Morinda citrifolia seeds were utilized. Solvents utilized for the extraction in this process were water and 80% ethanol. In alternative embodiments, other solvents may be utilized. In some embodiments, alcohol-based solvents may be utilized. In other embodiments, organic solvents may be utilized. For example, methanol or ethylacetate may be utilized as solvents. Similarly, the concentration of solvent may be modified. In the present example, 80% ethanol was utilized, however, in other embodiments, 50% ethanol, 60% ethanol, 70% ethanol, 90% ethanol and 100% ethanol may be utilized. Similarly, the concentration of alternative solvents may be modified as desired. Auxiliary substances utilized were Acaciae Gummi Pulvis and Silica hydrocolloidalis. Other auxiliary substances may be utilized and are discussed at greater lengths in the proceeding sections of the specification.

Seeds utilized in the present example were milled prior to the extraction process, and subsequently subject to a four millimeter sieve. The milling process was executed at ambient temperature and a continuous batch size of 300-350 kilograms of seed per hour was maintained. In some embodiments, larger or smaller batch sizes may be produced. For example, in some embodiments 500 kilograms of seed per hour may be processed, 1000 kilograms of seed per hour may be processed and/or 2000 kilograms of seed per hour may be processed. Alternatively, in some embodiments smaller batch sizes may be produced. For example, 5-100 kilograms of seed per hour may be produced, 100-150 kilograms of seed per hour may be produced and 150-300 kilograms of seed per hour may be produced.

In this preferred embodiment, the milled seeds were held in steel tanks at ambient temperature for a maximum of five days. In alternative embodiments, the seeds may be held for a shorter or longer period of time, and at different temperatures. For example, in some embodiments seeds may be held for 1-5 days. In other embodiments, the seeds may be held for 5-30 days, 30-60 days, 60-120 days, 120-360 days and 360-720 days. As previously mentioned, the storage temperature may likewise be modified as desired. In particular, the storage temperature may be 0-5° C., 5-15° C., 15-25° C., and 25-50° C.

Subsequently, according to one preferred embodiment, a primary alcohol extraction was performed. The primary extraction was conducted utilizing the milled seeds. In the present example, the primary extraction was performed utilizing 80% ethanol with a drug solvent ration of 1/6, at a temperature of 30-40° C. for 120 minutes. As previously mentioned, the percent of solvent utilized and the type of solvent utilized may be varied depending upon the desired result. Likewise, the drug solvent ratio may be modified and the temperature for performing the extraction and duration of extraction may also be modified. For example, in some embodiments a drug solvent ration of 1-1, 1-2, 1-3, 1-4, 1-5, 1-7, 1-8, 1-9, 1-10, 1-11, 1-12, 1-13, 1-14, 1-15, 1-16, 1-17, 1-18, 1-19, and 1-20 may be utilized. In some embodiments, the extraction may be carried out at 0-10° C., 10-20° C., 20-30° C., 40-50° C., 50-60° C., 70-80° C., and 80-100° C. Likewise, the duration of extraction in some embodiments is carried out for 1-5 minutes, 5-15 minutes, 15-25 minutes, 25-50 minutes, 50-80 minutes, 80-100 minutes, 100-120 minutes, 120-180 minutes, 180-240 minutes, and 240-300 minutes.

Subsequent to the extraction process, a separation of the extract from the drug residue was performed. Under the present example, the slurry of ethanol and milled seeds was pumped from the extraction vessel into a filter press and pressed out at an ambient temperature (maximum 35° C.) for a duration of 90 to 120 minutes. Various temperatures and durations may be utilized during the separation process. In some embodiments, a temperature lower than 35° C. is maintained. In other embodiments, a higher than 35° C. temperature is maintained for the duration of separation. For example, the separation process may occur at 0-10° C., 10-20° C., 20-30° C., 30-40° C., 40-50° C., 50-60° C., 60-70° C., 70-80° C., 80-90° C., and 90-100° C. Likewise, in some embodiments the duration of separation may be modified. In some embodiments, a period of time less than 90 minutes may be utilized and in some embodiments a period of time greater than 120 minutes may be utilized. For example, in some embodiments, the separation process may occur for 1-10 minutes, 10-20 minutes, 20-30 minutes, 30-40 minutes, 40-50 minutes, 50-60 minutes, 60-70 minutes, 70-80 minutes, 80-90 minutes, 90-100 minutes, 100-110 minutes, 110-120 minutes, 120-130 minutes, 130-140 minutes, 140-150 minutes, 150-160 minutes, 160-170 minutes, 170-180 minutes, 180-190 minutes, 190-200 minutes, 200-210 minutes, 210-220 minutes, 220-230 minutes, 230-240 minutes, 240-250 minutes, 250-260 minutes, 260-270 minutes, 270-280 minutes, 280-290 minutes, 290-300 minutes, 300-310 minutes, 310-320 minutes, 320-330 minutes, 330-340 minutes, 340-350 minutes, and 350-360 minutes. In the present example, the separated ethanol extract was temporarily stored at ambient temperature for a maximum of three days. Alternatively, the storage time and temperature of storage may be modified as previously suggested. Batch sizes of 10,000 kilograms of liquid extract were retained.

Subsequently, the extract was combined with an auxiliary agent. In the present example, a portion of Acaciae Gummi Pulvis was measured out in order to obtain 29% w/w related to the final dry product. In other embodiments, different final dry products may be desired. In particular, in some embodiments a 1-5% w/w final dry product may be desired. Alternatively, final dry products may be produced, according to some embodiments of the present invention, between 5-10% w/w, 10-20% w/w, 20-30% w/w, 30-40% w/w, 40-50% w/w, 50-60% w/w, 60-70% w/w, 70-80% w/w, and 80-90% w/w. The Acaciae Gummi Pulvis was then dissolved into hot water for extraction purposes to obtain a 10-20% w/w solution, at a dilution temperature of 80-90° C. In alternative preferred embodiments, the Acaciae Gummi Pulvis was utilized for mixing with the extraction product without first being mixed with hot water. Subsequently, a vessel was filled with approximately 1600-1800 kilograms of water. In other embodiments, less than 1600 kilograms of water may be utilized, and in other additional embodiments more than 1800 kilograms of water may be utilized. Accordingly, 1600-1800 kilograms of water represents only a non-limiting example of the amount of water utilized to mix the extract with an auxiliary substance. Additionally, solvents other than water may be utilized. In other embodiments, greater than 70% dry content is desired and in some embodiments less than 60% dry content is desired. Accordingly, a broad range of dry content is contemplated by the present invention.

Under preferred embodiments, the prepared Acaciae Gummi Pulvis may initially be added to the vessel. Once the evaporation process has been initiated, the filtrate from the Morinda citrifolia ethanol extract filtrate may then be continuously added to the evaporation circuit. The evaporation process was stopped when all of the filtrate was introduced and the desired dry content was reached. In some embodiments a dry content of 20-70% w/w is preferred. In preferred embodiments a dry content of the product after evaporation si 29% w/w.

Significant and unexpected advantages were experienced in some preferred embodiments of the extraction process and manufacturing regime as indicated above when the gum arabic is added to the tank initially, and then a continuous feed of the liquid extract is added so that there is always an excess of gum arabic in the mixture until the very end, at which point, the desired concentration is produced. When alternative processing methods were utilized, less effective results were experienced. A first unexpected benefit that was achieved was that the inhibition properties of the extract were substantially increased. Conversely, the inhibition properties of the extract were substantially diminished when gum arabic was continuously fed at a slow rate into a complete batch of the extract.

Further, precipitate formed when gum arabic was slowly added to a batch. Formation of precipitate during large scale processing has the added detrimental effect of baking the precipitate on to the heat exchangers during evaporation. Accordingly, maintaining the homogeneous nature of the fluid extract during large scale processing by continuously adding the extract to a homogenous premeasured mixture of an auxiliary agent produced unexpected efficacy and improved the manufacturing process. Accordingly, preferred embodiments of the present invention utilize the method as outlined above, wherein gum arabic is initially added to water and extract is slowly added into combination with the gum arabic. Utilizing this preferred method, no precipitate was visualized and an unexpected increase in inhibition was recorded in experiments conducted, as is shown in Example 1.

In some embodiments, after the extract is combined with an auxiliary agent additional processing may occur. In preferred embodiments, the combined materials may be concentrated. In the present example, the combined materials were concentrated by evaporation. The evaporation concentration step was conducted using an extract temperature of 40-45° C., a heating temperature of 75-80° C., and under vacuum of 100-120 mBar for a duration depending on the batch size.

The present invention contemplates utilizing various evaporation temperatures and vacuum pressures. In particular, the present invention contemplates using evaporation temperatures between 50° C. and 75° C., and evaporation temperatures between 80° C. and 100° C. Further, the present invention contemplates utilizing alternative vacuum pressures. In particular, the present invention contemplates utilizing vacuum pressures less than 100 mBar and vacuum pressures greater than 120 mBar, including vacuum pressures of 60 mBar, 70 mBar, 80 mBar, 90 mBar, 130 mBar, 140 mBar, 150 mBar, 160 mBar, 170 mBar and 180 mBar.

In preferred embodiments, the evaporation concentration process results in the production of an extract (e.g., the combination of an ingredient from the milled Morinda citrifolia seeds and the Acaciae Gummi Pulvis). In this preferred embodiment, the prepared extract was temporarily stored in steel tanks for a maximum of one week at 3-8° C. In some embodiments, the prepared extract may be stored for a longer duration of time and at divergent temperatures. In some embodiments, the prepared extract may be stored for one week, two weeks, one month, three months, six months and/or one year. In some embodiments the prepared extract may be stored at −10° C. to 0° C., 0° C. to 3° C., 8° C. to 15° C., and 15° C. to 25° C.

In some embodiments, the extract may be heat treated to prevent microbiological contamination. In preferred embodiments, the extract is ultra heat treated at a temperature of 100° C. for a duration of 30-40 seconds in batch sizes of 80-1500 kilograms. In alternative embodiments, the decontamination process may be carried out at alternative temperatures. For example, the decontamination temperature may be 110° C., 120° C., 150° C., 180° C. or 200° C. Likewise, in alternative embodiments, the duration of decontamination may be shorter and/or longer than the prior example. In particular, the present invention contemplates decontaminating the prepared materials for 5 to 10 seconds, 10 to 20 seconds, 20 to 30 seconds, 40 to 50 seconds, 50 to 60 seconds, 60 to 120 seconds, 120 to 240 seconds, and 240 to 360 seconds.

Subsequent to microbiological decontamination, the extract may be stored at a temperature of 3-8° C. for a maximum of two weeks. As indicated previously, the duration and temperature of storage may be modified as needed or as desired. In alternative embodiments, the temperature, pressure and duration of drying may all be modified. In some embodiments the drying temperature may be 25° C., 25-35° C., 35-45° C., 50-60° C., 60-70° C., 70-80° C., 80-90° C. and 90-100° C. Likewise, the vacuum pressure may be altered in various embodiments of the present invention. In some embodiments, the vacuum pressure may be maintained at range greater than 200 mBar or at range less than 150 mBar. In a non-limiting example, the vacuum pressure may be maintained between 100 and 120 mBar, 120-130 mBar, 130-140 mBar, 140-150 mBar, 200-250 mBar, and 250-300 mBar. Similarly, the duration of time required for drying may be changed. In some embodiments, the drying process may be conducted for 5-10 minutes, 10-20 minutes, 20-50 minutes, 50-70 minutes, 70-90 minutes, 120-180 minutes, 180-240 minutes, 240-300 minutes, and 300-360 minutes.

In some embodiments, the stored extract may be further processed by drying. In preferred embodiments, the stored extract may be vacuum belt dried at a temperature of 45-50° C. under a vacuum pressure of 150-200 mBar, for a duration of 90-120 minutes. Batch sizes may vary. In preferred embodiments, 15-20 kilograms of dried extract per run were produced, for a total production of 100-150 kilograms per day. In alternative embodiments, larger and smaller batch sizes may be produced. In particular, less than 15 kilogram batch sizes may be produced and greater than 20 kilogram batch sizes may be produced. In the non-limiting example, batch sizes may be selected from a list comprising 5 kilograms, 10 kilograms, 20 kilograms, 30 kilograms, 40 kilograms, 50 kilograms, 60 kilograms, 70 kilograms, 80 kilograms, 90 kilograms and 100 kilograms.

In some embodiments, the dried extract may be milled together with additional ingredients. In preferred embodiments, the dried extract is milled together with 1% w/w silica hydrocolloidalis at an ambient temperature maintaining relative air humidity below 40% for a duration of 5-10 minutes and sieved our through a 0.7 millimeter sieve. In alternative embodiments, more or less silica hydrocolloidalis may be utilized. Subsequently, additional steps may be taken to further process the prepared extract.

The present disclosure describes methods of preparing a Morinda citrifolia composition. This disclosure is descriptive only and not intended to be limiting. Many other embodiments of the invention will be recognized by one of skill in the art in light of the teachings of this disclosure. Therefore, the scope of the invention is determined by the appended claims.

What is claimed is:

1. A method for manufacturing a 5-Lipoxygenase and 15-Lipoxygenase inhibitor comprising the steps of:
collecting Morinda citrifolia seeds;
pulverizing the seeds;
adding the pulverized Morinda citrifolia seeds to an alcohol-based solution;
extracting an ingredient from said processed Morinda citrifolia seeds in solution to obtain a fraction;
inhibiting 5-Lipoxygenase and 15-Lipoxygenase by introducing said extracted ingredient to a mammal.

2. The method of claim 1, further comprising the step of combining the fraction with an auxiliary, by adding the extract over time to the entire amount of prepared carrier until the desired ratio of carrier to extract is reached.

3. The method of claim 2, wherein the auxiliary comprises gummi arabicum.

4. The method of claim 2, wherein the auxiliary comprises sterculia gummi.

5. The method of claim 2, wherein the auxiliary comprises methylcellulose.

6. The method of claim 2, further comprising the step of concentrating the fraction to a spissum by evaporation.

7. The method of claim 6, wherein the extract is concentrated until a dry content of 60-70% w/w is reached.

8. The method of claim 6, further comprising the step of drying the fraction to a dry extract.

9. The method of claim 6, wherein the concentration process is carried out at 75-80° C. under a vacuum pressure.

10. The method of claim 9, wherein the concentrating process is carried out at a vacuum pressure of 100-120 mBar.

11. The method of claim 1, wherein the alcohol-based solution is selected from a list consisting of ethanol and methanol and is present in an amount between about 30 and 96% by volume.

12. The method of claim 11, wherein the alcohol-based extraction is performed utilizing 80% ethanol in a drug solvent ratio of 1/6.

13. A composition for inhibiting 5-Lipoxygenase and 15-Lipoxygenase, said composition comprising a processed Morinda citrifolia component selected from a group consisting of extracts from Morinda citrifolia seeds, Morinda citrifolia seeds, and Morinda citrifolia extract combined with an auxiliary.

14. The composition of claim 13 produced in accordance with a method comprising the steps of:
collecting Morinda citrifolia seeds;
pulverizing the seeds;
adding the processed Morinda citrifolia seeds to an alcohol-based solution; and
extracting an ingredient from said processed Morinda citrifolia seeds in solution to obtain a fraction.

15. The composition of claim 14, further comprising the steps of combining the fraction with an auxiliary selected from a group consisting of gummi arabicum, sterculia gummi, and methylcellulose.

16. A method for isolating an active ingredient in a processed Morinda citrifolia product and utilizing said active ingredient to manufacture a Lipoxygenase inhibitor, said method comprising the step of:
obtaining an amount of seeds from a Morinda citrifolia plant;
combining seeds with an amount of an alcohol-based solution;
collecting an alcohol soluble fraction;
combining the fraction with an auxiliary selected from a group consisting of gummi arabicum, sterculia gummi, and methylcellulose; and
utilizing the combination of the fraction and auxiliary to prepare a nutraceutical formulation.

17. A method for manufacturing a COX-1, COX-2, Interleukin-1β, Interleukin-6, TNF-α, HLE, and iNOS inhibitor comprising the steps of:
collecting Morinda citrifolia seeds;
pulverizing the seeds;
adding the processed Morinda citrifolia seeds to an alcohol-based solution;
extracting an ingredient from said processed Morinda citrifolia seeds in solution to obtain a fraction;
inhibiting COX-1, COX-2, Interleukin-1β, Interleukin-6, TNF-α, HLE, and iNOS by introducing said extracted ingredient to a mammal.

18. The method of claim 17, further comprising the steps of combining the seeds with an auxiliary selected from the group consisting of gummi arabicum, sterculia gummi, and methylcellulose.

19. The method of claim 18, wherein the alcohol-based solution is selected from a list consisting of ethanol and methanol and is present in an amount between about 30 and 96% by volume.

20. The method of claim 17, wherein the alcohol based extraction is performed utilizing 80% ethanol in a drug solvent ratio of 1/6.

21. A composition for inhibiting COX-1, COX-2, Interleukin-1β, Interleukin-6, TNF-α, HLE, and iNOS, said composition comprising a processed Morinda citrifolia component selected from a group consisting of extracts from Morinda citrifolia seeds, Morinda citrifolia seeds, defatted pulverized Morinda citrifolia seed powder.

22. The composition of claim 21 produced in accordance with a method comprising the steps of:
collecting Morinda citrifolia seeds;
pulverizing the seeds;
adding the processed Morinda citrifolia seeds to an alcohol-based solution;
extracting an ingredient from said processed Morinda citrifolia seeds in solution to obtain a fraction.

23. A method for isolating an active ingredient in a processed Morinda citrifolia product and utilizing said active ingredient to manufacture a COX-1, COX-2, Interleukin-1β, Interleukin-6, TNF-α, HLE, and iNOS inhibitor, said method comprising the step of:
obtaining an amount of seeds from a Morinda citrifolia plant;
combining seeds with an amount of an alcohol-based solution;
collecting an alcohol soluble fraction;
combining the fraction with an auxiliary selected from the group consisting of gummi arabicum, sterculia gummi, and methylcellulose.
mixing said active ingredient into a naturaceutical formulation.
